(19) United States
(12) Patent Application Publication
(10) Pub. No.: US 2010/0241140 A1
(43) Pub. Date: Sep. 23, 2010

(54) Medical Clip with Bridge, System and Method of Using Same

(75) Inventor: Damian Jelich, Cottage Grove, MN (US)

Correspondence Address: Medtronic CardioVascular, Mounds View Facility South, 8200 Coral Sea Street N.E., Mounds View, MN, 55112, US

(73) Assignee: Medtronic, Inc., Minneapolis, MN (US)

(21) Appl. No.: 12/406,150

(22) Filed: Mar. 18, 2009

(51) Int. Cl. A61B 17/10; A61B 17/08

(52) U.S. Cl. 606/142; 606/213

(57) ABSTRACT

Device, system and method for drawing together patient tissue. A bridge segment has an axis and a first end and a second end opposite the first end. A first tine is coupled to the first end and projects from the bridge segment in a first direction orthogonal to the bridge segment. A second tine is coupled to the first end and projects from the bridge segment in a second direction approximately opposite the first direction. A third tine is coupled to the second end and projects from the bridge segment approximately in the first direction. A fourth tine is coupled to the second end and projects from the bridge segment approximately in the second direction. The first, second, third and fourth tines are each resiliently biased to form a coil approximately parallel to a plane orthogonal to the axis of the bridge segment.

### FIELD

The present invention relates generally to devices, systems and methods of drawing patient tissue together, and in particular such devices, systems and methods of drawing patient tissue together using a medical clip.

### BACKGROUND

In many circumstances and for many reasons, it is often beneficial to capture and draw together two or more pieces of tissue of a patient. The location of the tissue and the circumstances of the need to draw the pieces of tissue together have long resulted in a variety of different devices and methods for drawing the tissue together. Devices such as bandages, both self-adhesive and otherwise, clamps and stitches have been used to capture pieces of tissue and draw them together. Once tissue is drawn together, the natural healing abilities of the body may then allow the pieces of tissue to grow together, over time sealing a gap between the pieces of tissue such that the device is no longer needed. Alternatively, the pieces of tissue may be held together by the device indefinitely or for a particular period of time.

But, dependent on the location of the tissue, certain devices and methods may be impractical to utilize. For instance, while stitches may lend themselves well to readily accessible patient tissue, such as skin and muscle tissue, relatively inaccessible tissue, such as that found in the organs of the patient, may be impractical to capture and draw together using stitches. As such, the use of stitches to treat a defect in a patient's heart may tend require a traumatic open heart procedure, and even then, because the interior of the heart still may not be readily accessible, the treatment of a defect inside the heart may still not be attainable with stitches. Thus, a common device and method for drawing together patient tissue may not be applicable for all situations, particularly those involving a defect in organs of the patient such as the heart.

One relatively common defect in the heart of newborn children, which may also be present in older children and in adults, is a patent foramen ovale (“PFO”). During the gestation of a fetus in the womb, blood is oxygenated not by the undeveloped lungs of the fetus, but rather by the placenta of the mother. However, the heart of the fetus nevertheless pumps the blood through the cardiovascular system and receives the blood from the cardiovascular system. As such, in order to avoid the undeveloped lungs of the fetus, various vessels and bypasses exist that operate only during gestation that divert the blood from the lungs. At birth these bypasses typically close and circulation occurs by way of the lungs as with a normally developed adult.

An opening between the right atrium and the left atrium called the foramen ovale is open during gestation to prevent transfer of blood from the right ventricle of the heart to the lungs during gestation. Upon birth, the child's inherent circulation creates pressure within the atrium which causes a flap of tissue to close over the foramen ovale. As the child matures, the flap of tissue develops into a permanent closure. However, in some children the flap of tissue does not close, either in whole or in part, over the entire foramen ovale, creating a patent foramen ovale. The continued existence of the foramen ovale results in continued diversion from the lungs of at least some of the child's blood, reducing the flow of oxygenated blood through the child's system, and potentially leading to serious complications to the health of the child.

It is recognized that although PFO may occur most prominently in children and, in particular, relatively newborn children, that the PFO may also occur or be present in older children and in adults.

Other cardiac defects are known to exist beyond patent foramen ovales. For instance, atrial-septal defects (“ASD”) and ventricular-septal defects (“VSD”) likewise sometimes occur and may be detrimental to the health of the person, e.g., a child. Historically, open heart surgery had been required to fix such defects. But open heart surgery carries with it serious and well-known and recognized risks to the well-being of the person, in addition to being expensive and a considerable burden on hospital resources.

Closure devices for treating heart defects, such as patent foramen ovales, have been developed.

U.S. Pat. No. 6,776,784, Ginn, Clip Apparatus For Closing Septal Defects and Methods of Use, (Core Medical, Inc.) discloses a device for closing a septal defect, such as a patent foramen ovale, includes a clip formed from a superelastic material that is inserted into a septum wall of a heart. The clip is advanced through a patient's vasculature, e.g., within a delivery apparatus, until the clip is disposed within a first chamber adjacent the septal defect. Tines of the clip are directed through a flap of tissue of the septal defect until the tines of the clip are disposed within a second opposing chamber. The clip then transforms into its relaxed state, wherein the tines of the clip engage with a surface of the second chamber, thereby substantially closing the septal opening.

U.S. Patent Application Publication No. US2007/0060858, Sogard et al, Defect Occlusion Apparatus, System and Method, discloses occluding a multiplicity of parallel membranes, such as found in a patent foramen ovale. The methods, apparatus, and systems include the use of a positioning device that can be seated on the limbus of the septum secundum. The positioning device includes a piercing member that can pierce the septum secundum and septum primum. The positioning device also includes a fastening member that can engage the septum secundum and septum primum to fasten the tissues and thereby occlude a patent foramen ovale.

U.S. Pat. No. 7,220,265, Chanduszko et al, Patent Foramen Ovale (PFO) Closure Method and Device, (NMT Medical, Inc.) discloses methods and devices for closing two overlapping layers of tissue in a mammalian heart, such as a patent foramen ovale. The closure devices may take a number of different forms and may be retrievable. In some embodiments, the closure devices may be delivered with a catheter capable of puncturing mammalian tissue. In some embodiments, a spring-like bioabsorbable polymer material are used, in one such embodiment as a “grappling hook”, to embed in and draw together the pieces of tissue. In another embodiment, a suture is delivered, and an anchor forms a pre-determined shape and engages the septum secundum, closing the patent foramen ovale.

### SUMMARY

Closure devices for treating patent foramen ovales have been developed that allow for the treatment of patent foramen ovales and other cardiac defects without conducting open heart surgery. Instead, the closure devices may be utilized to cure or treat cardiac defects by way of transvenous implantation. With the device placed in a sheath attached to a catheter small enough to pass through the blood vessels of the child and into the heart, the device may be deployed in the heart to treat the cardiac defect.

The device itself may be made of a number of joined tines, each of which may form a loop, perhaps sharpened at the end to allow for puncturing the cardiac tissue of the patient. Outside of the implantation sheath each tine may be biased so that each tine forms a loop or coil in a relaxed state. Inside of the sheath the tines are uncoiled to be relative linear, possibly giving the device an adequately small profile to allow passage of the device through a vein or other vascular component of the patient. When the device is deployed from the sheath the tines may curl into a biased (relaxed) coiled form. During a transition from a relatively linear configuration to a coiled configuration, as the device is deployed from the sheath, within the heart the sharpened end of the tines may pass through cardiac tissue. In the case of the treatment of a patent foramen ovale, if the device is positioned adjoining the two flaps of tissue which did not automatically close together, at least one approximately linear tine may pass through each flap of tissue as coils are formed. As the tines complete forming a coil, the two flaps of tissue may be drawn together, either closing the foramen ovale altogether, or bringing the flaps of tissue in closer proximity of each other such that vascular pressure may ultimately bring the flaps of tissue together. As the patient matures flaps of tissue may grow together and the foramen ovale close permanently.

In an embodiment, a medical clip is disclosed for drawing together patient tissue. The medical clip comprises a bridge segment having an axis and a first end and a second end opposite the first end, a first tine coupled to the first end of the bridge segment and projecting from the bridge segment in a first direction approximately orthogonal to the bridge segment, a second tine coupled to the first end of the bridge segment and projecting from the bridge segment in a second direction approximately opposite the first direction, a third tine operatively coupled to the second end of the bridge segment and projecting from the bridge segment approximately in the first direction, and a fourth tine coupled to the second end of the bridge segment and projection from the bridge segment approximately in the second direction. The first tine, the second tine, the third tine and the fourth tine are each resiliently biased to form a coil approximately parallel to a plane orthogonal to the axis of the bridge segment.

In an embodiment, each of the tines may be flexed to be approximately linear.

In an embodiment, the coil comprises at least one full revolution.

In an embodiment, the coil comprises at least one-and-a-quarter revolutions.

In an embodiment, the tines are configured to pass through the patient tissue.

In an embodiment, the tines are configured to draw a first piece of the patient tissue together with a second piece of the patient tissue.

In an embodiment, at least one of the first tine and the third tine are configured to pass through the first piece of patient tissue and wherein at least one of the second tine and the fourth tine are configured to pass through the second piece of patient tissue.

In an embodiment, the first tine and the third tine pass through the first piece of patient tissue and the second tine and the fourth tine pass through the second piece of patient tissue during a transition from being approximately linear to forming the coil.

In an embodiment, the first piece of tissue is drawn together with the second piece of tissue when the tines form the coil.

In an embodiment, the first tine, the second tine, the third tine and the fourth tine are each comprised of nitinol.

In an embodiment, a system is disclosed for drawing together patient tissue. The system comprises a medical clip and a delivery catheter. The medical clip comprises a bridge segment having an axis and a first end and a second end opposite the first end, a first tine coupled to the first end and projecting from the bridge segment in a first direction approximately orthogonal to the bridge segment, a second tine coupled to the first end and projecting from the bridge segment in a second direction approximately opposite the first direction, a third tine operatively coupled to the second end and projecting from the bridge segment approximately in the first direction, and a fourth tine coupled to the second end and projection from the bridge segment approximately in the second direction. Each of the tines is resiliently biased to form a coil approximately parallel to a plane orthogonal to the axis of the bridge segment. The delivery catheter comprises a lumen containing the medical clip with the first tine, the second tine, the third tine and the fourth tine being flexed to be approximately linear by the delivery catheter and a deployment mechanism positioned within the lumen adapted to deploy the medical clip by pushing the medical clip out of an opening in the lumen. The first tine, the second tine, the third tine and the fourth tine coil when the medical clip is deployed from the delivery catheter.

In an embodiment, a method is disclosed for drawing together tissue of a patient using a medical clip having a first tine and a third tine coupled to a first end of a bridge segment and a second tine and a fourth tine coupled to a second end of the bridge segment, wherein the first tine, the second tine, the third tine and the fourth tine are each resiliently biased to form a coil. The method comprises the steps of placing a delivery catheter having a lumen containing the medical clip between a first piece of the tissue and a second piece of the tissue, wherein the first tine, the second tine, the third tine and the fourth tine are straightened while in the delivery catheter and deploying the medical clip from the delivery catheter. Upon coiling, the first tine and the third tine and pass through the first piece of tissue and upon coiling, the second tine and the fourth tine pass through the second piece of tissue. The first piece of tissue and the second piece of tissue are drawn together upon coiling of the first tine, the second tine, the third tine and the fourth tine.

In an embodiment, a method is disclosed for treating a patent foramen ovale using a medical clip having a first tine and a third tine coupled to a first end of a bridge segment and a second tine and a fourth tine coupled to a second end of the bridge segment, wherein the first tine, the second tine, the third tine and the fourth tine are each resiliently biased to form a coil. The method comprises the steps of placing a delivery catheter having a lumen containing the medical clip between a first piece of tissue of the patent foramen ovale and a second piece of tissue of the patent foramen ovale, wherein the first tine, the second tine, the third tine and the fourth tine are straightened while in the delivery catheter and deploying the medical clip from the delivery catheter. Upon coiling, the first tine and the third tine and pass through the first piece of tissue of the patent foramen ovale and, upon coiling, the second tine and the fourth tine and pass through the second piece of tissue of the patent foramen ovale. The patent foramen ovale is reduced in size when the first piece of tissue of the patent foramen ovale and the second piece of tissue of the patent foramen ovale are drawn together upon coiling of the first tine, the second tine, the third tine and the fourth tine.

In an embodiment, the patent foramen ovale is closed upon coiling of the first tine, the second tine, the third tine and the fourth tine coil.

In an embodiment, a method is disclosed for making a system for drawing together patient tissue using a medical clip. The method comprises the steps of forming the medical clip by coupling a plurality of tines to a bridge segment, each individual one of the plurality of tines being resiliently biased to form a coil, inserting the medical clip into a lumen of a delivery catheter having a distal end such that the plurality of tines are oriented toward the distal end, wherein the plurality of tines uncoil and become approximately linear upon each individual one of the plurality of tines entering the lumen, inserting a deployment mechanism in the lumen of the delivery catheter such that using the deployment mechanism deploys the medical clip out of an end of the lumen. The plurality of tines begin to coil upon being deployed, penetrating the patient tissue and drawing the patient tissue together as each individual one of the plurality of tines form a coil.

In an embodiment, the inserting the medical clip into the lumen step comprises inserting the bridge segment into the lumen of the delivery catheter and drawing the bridge segment down the lumen at least as far as a length of individual ones of the plurality of tines.

In an embodiment, the deployment mechanism pushes on the medical clip to deploy the medical clip.

### DRAWINGS

FIG. 1 shows a medical clip for drawing together patient tissue;

FIG. 2 shows the medical clip of FIG. 1 positioned in a deployment catheter;

FIG. 3 shows the medical clip of FIG. 1 deployed from, but still coupled to the deployment catheter;

FIGS. 4a-4c illustrate the positioning and deployment of the medical clip of FIG. 1 for the treatment of a patent foramen ovale;

FIG. 5 is a flowchart for implanting a medical clip;

FIG. 6 is a flowchart for making a system including a medical clip positioned in a deployment catheter;

FIG. 7 shows the medical clip of FIG. 1 deployed from the deployment catheter with an alternative deployment mechanism; and

FIG. 8 is a flowchart for explanting a medical clip.

### DESCRIPTION

It is often advantageous to capture and draw together pieces of tissue of a patient. Doing so may close and help wounds heal, or close defects in patient organs. Various capture and closure devices exist, but while such devices may be effective in certain situations and under certain conditions, they may be ineffective or disadvantageous in other conditions. Particularly in situations where the tissue to be drawn together is not readily accessible to personal manipulation, commonly known devices are often of limited use. The treatment of cardiac defects may be one such relatively common situation.

In order to treat cardiac defects such as a patent foramen ovale, it is desirable close the gap between flaps of cardiac tissue without having to experience the trauma and expense of open heart surgery. Accordingly, a medical clip and delivery system has been developed that may be inserted into the heart intravenously. Upon positioning the delivery system within the gap of the patent foramen ovale, a deployment system deploys the medical clip. The physical nature of the medical clip may capture and draw together the flaps of tissue of the patent foramen ovale, thereby reducing or closing the gap between the pieces of tissue altogether.

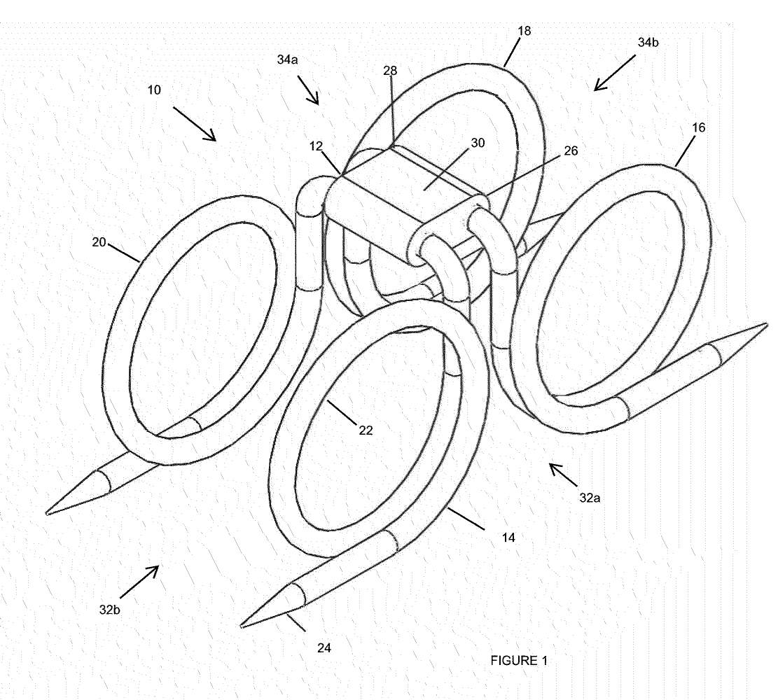

An embodiment of a medical clip for treating cardiac defects is illustrated in FIG. 1. Clip 10 has bridge section 12. Four tines 14, 16, 18, 20 project from bridge section 12. Each tine 14, 16, 18, 20 is comprised of coil 22 and end 24. In an embodiment, end 24 forms a sharpened tip. In the illustrated embodiment, tines 14, 16, 18, 20 are coupled to bridge section 18, with two tines 14, 16 coupled to end 26 and two tines 18, 20 coupled to end 28. In the illustrated embodiment, bridge section 12 has bulk 20 to which the tines 14, 16, 18, 20 are coupled.

In various alternative embodiments, the number of tines 14, 16, 18, 20 may be altered based on various criteria. For instance, in some circumstances it may be desirable to have six total tines, such as when an amount of tissue to be captured is relatively large. Increases to eight or more tines may likewise be desirable. Further, it may be advantageous to utilize unequal numbers of tines on opposing sides of clip 10, for instance when the flaps of tissue to be drawn together are of unequal size. In one embodiment, one side may have two tines while the opposite side may have one tine. Various other combinations of numbers of tines are envisioned.

In an alternative embodiment, tine 14 and tine 16 are made from a single length of material, and tine 18 and tine 20 are made from a single length of material, forming two tine pairs 32a, 34a, with each tine pair 32a, 34a then being coupled to bridge section 12. In another alternative embodiment, tine 14 and tine 20 are made from a single length of material, and tine 16 and tine 18 are made from a single length of material, to form two tine pairs 32b, 34b. Tine pairs 32b, 34b may then be coupled together at bridge section 12. Coupling may be accomplished utilizing a weld, adhesive, or by any means of joining the pairs 32a, 32b, 34a, 34b suitable for use in a human body. Alternatively, bulk 30 may be coupled to each tine pair 32b, 34b, forming clip 10 and bridge section 12 without coupling tine pairs 32b, 34b together directly.

Tines 14, 16, 18, 20 may be made from a variety of different materials. Any material may be used such that tines 14, 16, 18, 20 may be resiliently biased to form coil 22 when clip 10 has been deployed, provided the material is biocompatible or may be treated to make it biocompatible. In an embodiment, tines 14, 16, 18, 20 are made from the shaped memory alloy Nitinol. In alternative embodiments, biocompatible elastic material such as stainless steel may be utilized. Biocompatible super-elastic materials may also be utilized. Super-elastic materials could encompass super-elastic plastics and super-elastic metals. A super-elastic plastic generally is any material that has shape memory ability after shaped setting, e.g., materials described in the Massachusetts Institute of Technology, News Office article entitled “Intelligent Plastics Change Shape With Light, dated Apr. 13, 2005, authored by Elizabeth A. Thomson, which is hereby incorporated by references in its entirety. Super-elastic metals are sometimes known as a shape memory alloy (also, smart metal, memory alloy or muscle wire) that remembers its shape and can be returned to that shape after being deformed, by applying heat to the alloy. When the shape memory effect is correctly harnessed, super-elastic metals becomes a lightweight, solid-state alternative to conventional actuators such as hydraulic, pneumatic and motor-based systems. In an embodiment, drawn filled tubes filled with a super-elastic material or materials.

In a further alternative embodiment, spring-like bioabsorbable material may be utilized, which may result in clip 10 ultimately dissolving. Alternatively, a non-bioabsorbable material may be utilized to form clip 10, but the material may be coated with biological tissue, bioabsorbable polymer, a therapeutic substance or other substance which may be advantageously delivered to the treatment site concurrent with clip 10.

In the illustrated embodiment of FIG. 1, coils 22 create a full circular loop, whereby a completed circle is formed because individual tines 14, 16, 18, 20 complete an approximately circular circuit. As illustrated, tines 14, 16, 18, 20 complete one-and-a-quarter revolutions, with the overlap providing potentially enhanced ability to hold tissue over a coil which completes only one full revolution. Alternatively, coils 22 may form only a partial circular loop. In an embodiment, coil 22 may be only three-quarters of a completed circle. In such an embodiment, the tissue captured in coil 22 may be sufficiently secured that the flaps of tissue may be drawn together even without coil 22 forming a completed circle. Alternative partial loops may also be utilized such that the tissue may still be captured and retained by coil 22. In further alternative embodiments, non-circular loops may also be utilized. Oval or ellipsoid shapes may be utilized advantageously in certain circumstances. Alternatively, shapes with angles such as triangles or rectangles may be utilized. Further, irregular shapes may be utilized.

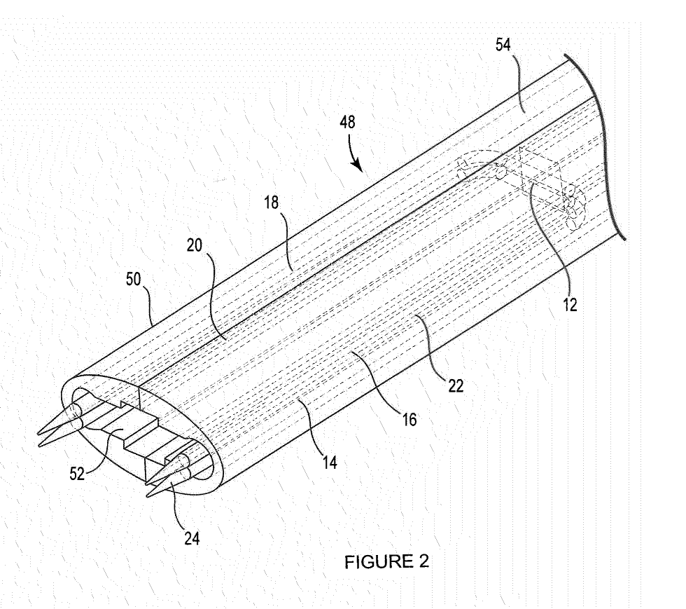

FIG. 2 illustrates an embodiment of clip 10 in an embodiment of a deployment catheter 48 having a sheath 50. To place clip 10 in sheath 50, bridge section 12 may be drawn into lumen 52 of sheath 50. As bridge section 12 is drawn in to sheath 50, coil 22 of each tine 14, 16, 18, 20 is stressed by sheath 50, causing coil 22 to unwind and each tine 14, 16, 18, 20 to become approximately straight when positioned in sheath 50. End 24 of each tine 14, 16, 18, 20 remains in proximity of the distal end of sheath 50.

Sheath 50 may be coupled to catheter 54 to form deployment catheter 48. Catheter 54 may be utilized to guide sheath 50 into position to deploy clip 10. Catheter 54 may also be utilized to deploy clip 10. In an embodiment, catheter 54 has a deployment mechanism 56 in contact with bridge section 12. When deployment mechanism 56 pushes bridge section 12, clip 10 slides along sheath 50 until clip 10 has fully emerged from sheath 50.

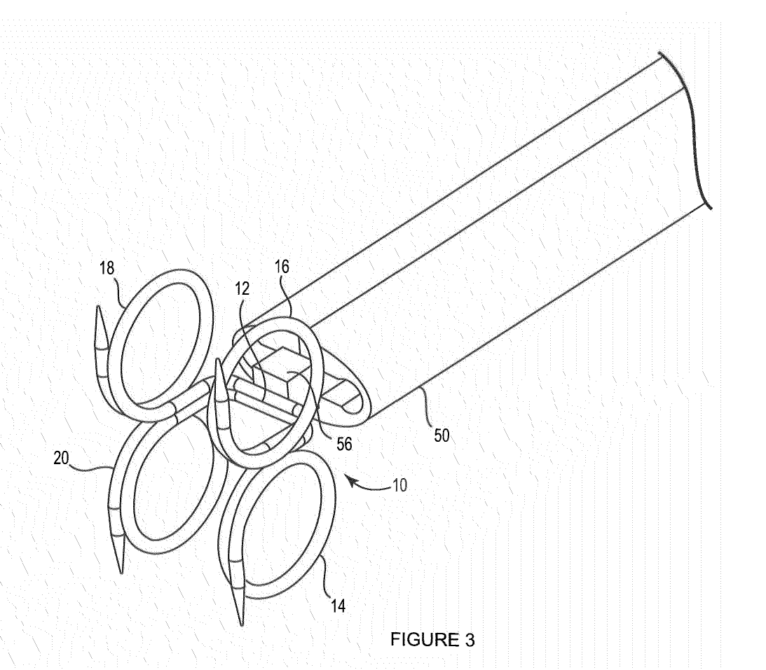

FIG. 3 illustrates an embodiment of clip 10 fully emerged from sheath 50 but still attached to deployment mechanism 56. In the illustrated embodiment, tines 14, 16, 18, 20 have returned to their resiliently biased coiled state. In various embodiments of clip 10, dependent on the material from which tines 14, 16, 18, 20 were made, tines 14, 16, 18, 20 may coil within moments of emerging from sheath 50, or may require a lengthier amount of time to coil. In certain embodiments, tines 14, 16, 18, 20 may, for a time, remain essentially straight even after bridge section 12 has emerged from sheath 50 due to the length of time required to coil. However, in such an embodiment, tines 14, 16, 18, 20 will eventually coil after clip 10 has deployed from sheath 50.

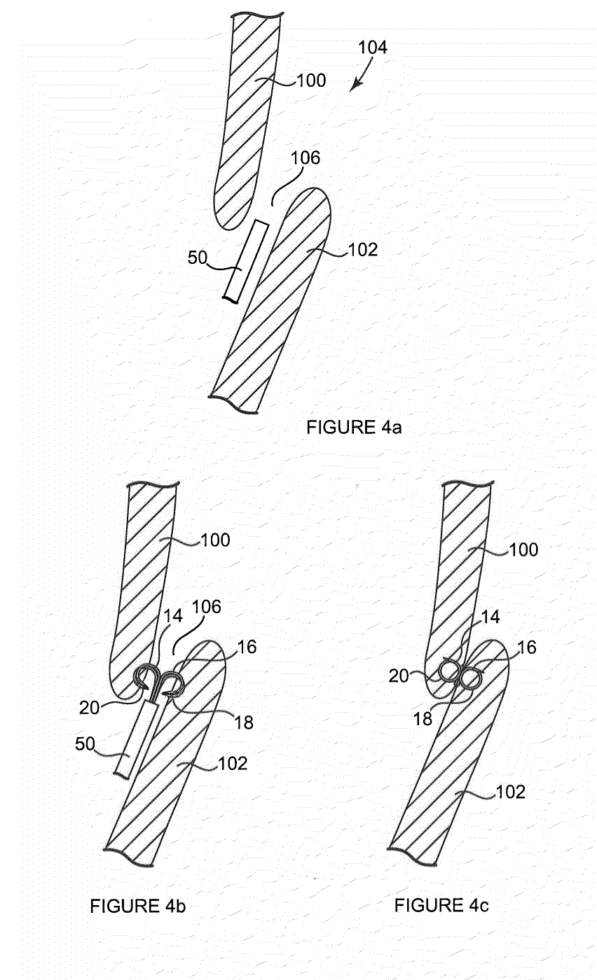

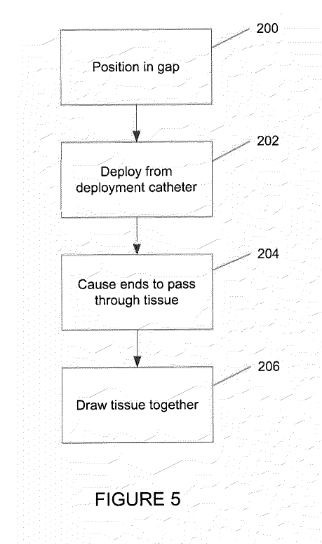

FIGS. 4a-4c and the flowchart of FIG. 5 illustrate a use of clip 10 and sheath 50 in the treatment of a patent foramen ovale. Sheath 50 is positioned (FIG. 5, 200) in a gap 106 between a first flap of tissue 100 and a second flap of tissue 102 within heart 104 while clip 10 remains in sheath 50 with tines 14, 16, 18, 20 uncoiled (FIG. 4a). As clip 10 is deployed (FIG. 5, 202) from sheath 50, tines 14, 16, 18, 20 begin to coil, with the coiling motion causing (FIG. 5, 204) end 24 of tine 14 and tine 20 to pass through tissue 100 and end 24 of tine 16 and tine 18 passing through tissue 102 (FIG. 4b). As tines 14, 16, 18, 20 complete coiling, tissue 100 and tissue 102 are drawn (FIG. 5, 206) together by the decrease in the radius of coil 22 as each tine 14, 16, 18, 20 coils. The completion of coiling may draw tissue 100 and tissue 102 completely together and close gap 106 (FIG. 4c). Alternatively, the completion of coiling may draw tissue 100 and tissue 102 nearly together though still leaving a reduced gap 106.

The treatment of other cardiac defects, such as atrial-septal defects (“ASD”) and ventricular-septal defects (“VSD”), by the same steps illustrated above is also contemplated. In fact, any patient condition in which it is desirable to join or draw together two flaps of tissue may be effectively treated utilizing the steps illustrated in FIGS. 4a-4c. While the physical dimensions of clip 10 and delivery catheter 54 may need to change to reflect the different conditions, such as a wider gap 106 or tougher or thicker tissue 100, 102, the method of using clip 10 may remain unchanged.

In various implementations of the deployment of clip 10, tissue 100 and tissue 102 may be drawn together even if one or two of tines 14, 16, 18, 20 do not pass through tissue as intended. So long as at least one tine 14, 16, 18, 20 passes through each flap of tissue 100, 102, tissue 100 may be drawn together with tissue 102. Instances in which all four tines 14, 16, 18, 20 pass through tissue 100, 102 may, however, create the highest likelihood of maximizing the amount of tissue 100, 102 brought together.

The geometry of the components of clip 10 influence performance of clip 10. Relatively longer tines 14, 16, 18, 20 and relatively greater diameter of coil 22 may allow for the treatment of a relatively larger gap 106, or result in greater depth of penetration of tissue 100, 102, perhaps increasing the likelihood of closing gap 106 altogether. A relatively longer length of bridge section 12 may result in an increased area of tissue 100, 102 that is drawn together, thereby increasing the tissue area brought together, aiding in the ultimate sealing of the foramen ovale. And a relatively greater diameter of the material comprising tines 14, 16, 18, 20 may increase the ability of tines 14, 16, 18, 20 to hold tissue 100, 102 and decrease the likelihood of tissue 100, 102 slipping out of tines 14, 16, 18, 20 during or after coiling. In an embodiment, the dimensions of coil 22 is approximately 0.090 inches, the length of bridge section 12 is 0.070 inches, and the diameter of tines 14, 16, 18, 20 is 0.014 inches. In alternative embodiments, coil 22 may range from 0.050 inches to 0.125 inches, bridge section 12 may range from 0.050 inches to 0.25 inches, and the diameter of times 14, 16, 18, 20 may range from 0.008 inches to 0.018 inches.

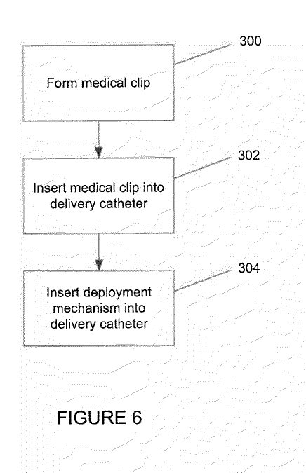

FIG. 6 is a flowchart of a method for making a system in which an embodiment of medical clip 10 is positioned in deployment catheter 48. Medical clip 10 is formed (300) by coupling tines 14, 16, 18, 20 to bridge section 12. Medical clip 10 is inserted (302) into lumen 52 of delivery catheter 48. In an embodiment, bridge section 12 is inserted into lumen 52 first, followed by tines 14, 16, 18, 20. As the insertion occurs, tines 14, 16, 18, 20 uncoil until they are approximately linear within lumen 52. Deployment mechanism 56 is inserted (304) into lumen 52, such that when the deployment mechanism is used, medical clip 10 deploys out of lumen 52 and tines 14, 16, 18, 20 coil. In embodiments where deployment mechanism 56 is coupled to medical clip 10, coupling deployment mechanism 56 to medical clip 10 may occur either before or after insertion of medical clip into delivery catheter 48. When coupling occurs before insertion of medical clip 10, deployment mechanism 56 may be utilized to insert medical clip 10 into deployment catheter 48 by pulling medical clip into lumen 52.

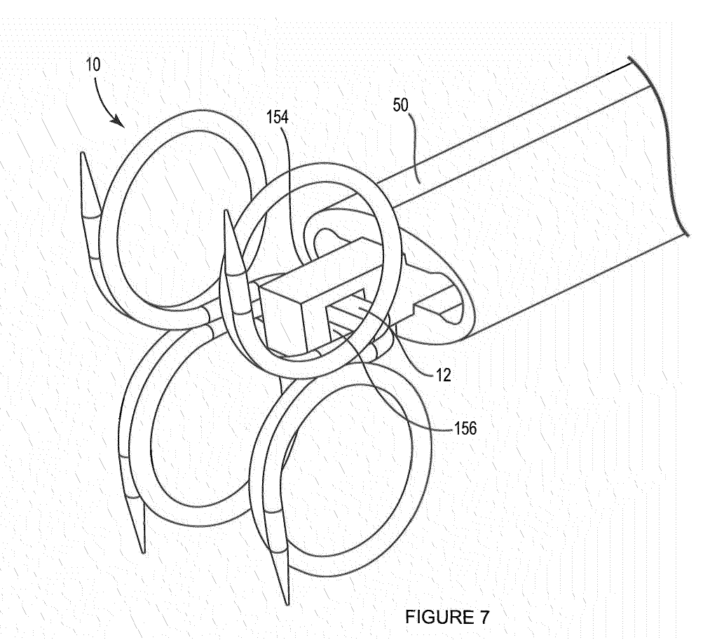

FIG. 7 illustrates an alternative embodiment of deployment mechanism 154. In contrast with deployment mechanism 54 (FIGS. 2 and 3), which may deploy medical clip 10 simply by pushing on medical clip 10 until it emerges from sheath 50, deployment mechanism 154 may engage bridge section 12 in notch 156. With bridge section 12 deployed in notch 156, medical clip 10 may be deployed from sheath 50, or may be retracted back into sheath 50 by engaging medical clip 10, e.g., bridge segment 12, in notch 156 and drawing deployment mechanism 154 back into sheath 50. In various embodiments, deployment mechanism 154 may be utilized to secure and retract medical clip 10 after medical clip 10 has been fully deployed into tissue 100, 102 (FIG. 4) by engaging bridge section 12 in notch 156 and exerting a pulling force using deployment mechanism 154. Alternative embodiments of deployment mechanism 154 may utilize clamps, magnets, or any other selectively engageable fastening mechanism to engage medical clip 10 with deployment mechanism 154 such that medical clip 10 may be retracted into sheath 50.

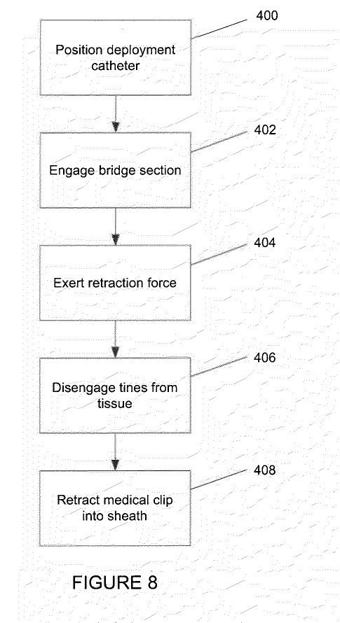

FIG. 8 is a flow chart of a method of explanting a medical clip 10 deployed in tissue 100, 102, as illustrated in FIG. 4c. Deployment catheter 48 is positioned (400) in proximity of bridge section 12 of medical clip 10. Deployment mechanism 154 engages (402) bridge section 12. As retraction force is exerted (404) on deployment mechanism 154 bridge section 12 moves away from tissue 100, 102 and tines 14, 16, 18, 20 begin uncoiling. Ultimately, tines 14, 16, 18, 20 disengage (406) from tissue 100, 102 and medical clip may be fully retracted (408) into sheath 50.

Thus, embodiments of the devices, system and methods of drawing patient tissue together are disclosed. One skilled in the art will appreciate that the present invention can be practiced with embodiments other than those disclosed. The disclosed embodiments are presented for purposes of illustration and not limitation, and the present invention is limited only by the claims that follow.

What is claimed is:

1. A medical clip for drawing together patient tissue, comprising:
a bridge segment having an axis and a first end and a second end opposite said first end;
a first tine coupled to said first end of said bridge segment and projecting from said bridge segment in a first direction approximately orthogonal to said bridge segment;
a second tine coupled to said first end of said bridge segment and projecting from said bridge in a second direction approximately opposite said first direction;
a third tine operatively coupled to said second end of said bridge segment and projecting from said bridge approximately in said first direction; and
a fourth tine coupled to said second end of said bridge segment and projection from said bridge segment approximately in said second direction;
said first tine, said second tine, said third tine and said fourth tine each being resiliently biased to form a coil approximately parallel to a plane orthogonal to said axis of said bridge segment.

2. The medical clip of claim 1 wherein each of said tines may be flexed to be approximately linear.

3. The medical clip of claim 1 wherein said coil comprises at least one full revolution.

4. The medical clip of claim 3 wherein said coil comprises at least one-and-a-quarter revolutions.

5. The medical clip of claim 1 wherein said tines are configured to pass through said patient tissue.

6. The medical clip of claim 2 wherein said tines are configured to draw a first piece of said patient tissue together with a second piece of said patient tissue.

7. The medical clip of claim 6 wherein at least one of said first tine and said third tine are configured to pass through said first piece of patient tissue and wherein at least one of said second tine and said fourth tine are configured to pass through said second piece of patient tissue.

8. The medical clip of claim 7 wherein said first tine and said third tine pass through said first piece of patient tissue and said second tine and said fourth tine pass through said second piece of patient tissue during a transition from being approximately linear to forming said coil.

9. The medical clip of claim 8 wherein said first piece of tissue is drawn together with said second piece of tissue when said tines form said coil.

10. The medical clip of claim 1 wherein said first tine, said second tine, said third tine and said fourth tine are each comprised of nitinol.

11. A system for drawing together patient tissue, comprising:
a medical clip, comprising:
a bridge segment having an axis and a first end and a second end opposite said first end;
a first tine coupled to said first end and projecting from said bridge in a first direction approximately orthogonal to said bridge segment;
a second tine coupled to said first end and projecting from said bridge in a second direction approximately opposite said first direction;
a third tine operatively coupled to said second end and projecting from said bridge approximately in said first direction; and
a fourth tine coupled to said second end and projection from said bridge approximately in said second direction;
wherein each of said tines is resiliently biased to form a coil approximately parallel to a plane orthogonal to said axis of said bridge segment; and
a delivery catheter, comprising:
a lumen containing said medical clip with said first tine, said second tine, said third tine and said fourth tine being flexed to be approximately linear by said delivery catheter;
a deployment mechanism positioned within said lumen adapted to deploy said medical clip by pushing said medical clip out of an opening in said lumen;
wherein said first tine, said second tine, said third tine and said fourth tine coil when said medical clip is deployed from said delivery catheter.

12. The system of claim 11 wherein each of said tines may be flexed to be approximately linear.

13. The system of claim 11 wherein said coil comprises at least one full revolution.

14 The system of claim 13 wherein said coil comprises at least one-and-a-quarter revolutions.

15. The system of claim 11 wherein said tines are configured to pass through said patient tissue.

16. The system of claim 12 wherein said tines are configured to draw a first piece of said patient tissue together with a second piece of said patient tissue.

17. The system of claim 16 wherein at least one of said first tine and said third tine are configured to pass through said first piece of patient tissue and wherein at least one of said second tine and said fourth tine are configured to pass through said second piece of patient tissue.

18. The system of claim 17 wherein said first tine and said third tine pass through said first piece of patient tissue and said second tine and said fourth tine pass through said second piece of patient tissue during a transition from being approximately linear to forming said coil.

19. The system of claim 18 wherein said first piece of tissue is drawn together with said second piece of tissue when said tines form said coil.

20. The system of claim 1 wherein said first tine, said second tine, said third tine and said fourth tine are each comprised of nitinol.

21. A method for drawing together tissue of a patient using a medical clip having a first tine and a third tine coupled to a first end of a bridge segment and a second tine and a fourth tine coupled to a second end of said bridge segment, wherein said first tine, said second tine, said third tine and said fourth tine are each resiliently biased to form a coil, comprising the steps of:
placing a delivery catheter having a lumen containing said medical clip between a first piece of said tissue and a second piece of said tissue, wherein said first tine, said second tine, said third tine and said fourth tine are straightened while in said delivery catheter;
deploying said medical clip from said delivery catheter;
wherein, upon coiling, said first tine and said third tine and pass through said first piece of tissue and wherein, upon coiling, said second tine and said fourth tine pass through said second piece of tissue; and
wherein said first piece of tissue and said second piece of tissue are drawn together upon coiling of said first tine, said second tine, said third tine and said fourth tine.

22. A method for treating a patent foramen ovale using a medical clip having a first tine and a third tine coupled to a first end of a bridge segment and a second tine and a fourth tine coupled to a second end of said bridge segment, wherein said first tine, said second tine, said third tine and said fourth tine are each resiliently biased to form a coil, comprising the steps of:
placing a delivery catheter having a lumen containing said medical clip between a first piece of tissue of said patent foramen ovale and a second piece of tissue of said patent foramen ovale, wherein said first tine, said second tine, said third tine and said fourth tine are straightened while in said delivery catheter;
deploying said medical clip from said delivery catheter;
wherein, upon coiling, said first tine and said third tine and pass through said first piece of tissue of said patent foramen ovale and wherein, upon coiling, said second tine and said fourth tine and pass through said second piece of tissue of said patent foramen ovale; and
wherein said patent foramen ovale is reduced in size when said first piece of tissue of said patent foramen ovale and said second piece of tissue of said patent foramen ovale are drawn together upon coiling of said first tine, said second tine, said third tine and said fourth tine.

23. A method as in claim 22 wherein said patent foramen ovale is closed upon coiling of said first tine, said second tine, said third tine and said fourth tine coil.

24. A method of making a system for drawing together patient tissue using a medical clip, comprising the steps of:
forming said medical clip by coupling a plurality of tines to a bridge segment, each individual one of said plurality of tines being resiliently biased to form a coil;
inserting said medical clip into a lumen of a delivery catheter having a distal end such that said plurality of tines are oriented toward said distal end, wherein said plurality of tines uncoil and become approximately linear upon each individual one of said plurality of tines entering said lumen;
inserting a deployment mechanism in said lumen of said delivery catheter such that using said deployment mechanism deploys said medical clip out of an end of said lumen;
whereby said plurality of tines begin to coil upon being deployed, penetrating said patient tissue and drawing said patient tissue together as each individual one of said plurality of tines form a coil.

32. A method as in claim 31 wherein said inserting said medical clip into said lumen step comprises inserting said bridge segment into said lumen of said delivery catheter and drawing said bridge segment down said lumen at least as far as a length of individual ones of said plurality of tines.

33. A method as in claim 31 wherein said deployment mechanism pushes on said medical clip to deploy said medical clip.
